(19) United States
(12) Patent Application Publication
(10) Pub. No.: US 2024/0001037 A1
(43) Pub. Date: Jan. 4, 2024

(54) PLUNGER TIP COVER WITH LOCKING MEMBERS, SYSTEMS, AND METHODS

(71) Applicant: Merit Medical Systems, Inc., South Jordan, UT (US)

(72) Inventors: Richard P. Jenkins, Bluffdale, UT (US); Gregory R. McArthur, Sandy, UT (US)

(21) Appl. No.: 18/344,180

(22) Filed: Jun. 29, 2023

Related U.S. Application Data
(60) Provisional application No. 63/367,520, filed on Jul. 1, 2022.
(60) Provisional application No. 63/385,933, filed on Dec. 2, 2022.

(51) Int. Cl. A61M 5/315

(52) U.S. Cl. CPC A61M 5/31513; A61M 5/31571

(57) ABSTRACT

Coverings for syringe plunger tips, as well as syringes and syringe components including such coverings, are disclosed. A material of the covering may have at least one different material property than a material of the plunger tip. The coverings may provide a reduced coefficient of friction between the covering and a surface of the syringe relative to the coefficient of friction between the plunger tip and the syringe surface. The coverings may also isolate a substance loaded in the syringe from the plunger tip. The coverings may include a locking member to lock the covering over the plunger tip. Methods for manufacturing a syringe assembly including such coverings are also disclosed.

### RELATED CASES

This application claims priority to U.S. Provisional Application No. 63/367,520, filed on Jul. 1, 2022 and titled “PLUNGER TIP COVER WITH LOCKING MEMBERS FOR SILICONE-FREE SYRINGE BARREL”, and U.S. Provisional Application No. 63/385,933, filed on Dec. 2, 2022 and titled “PLUNGER TIP COVER WITH LOCKING MEMBER, SYSTEMS, AND METHODS”, both of which are hereby incorporated by reference in their entirety.

### TECHNICAL FIELD

The present disclosure relates to the field of medical devices. More specifically, the present disclosure relates to a syringe having a syringe plunger and a syringe plunger tip. Even more specifically, the present disclosure relates to various coverings for syringe plunger tips and methods related thereto.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings, in which:

FIG. 1A illustrates a perspective view of an embodiment of a syringe assembly according to the present disclosure.

FIG. 1B illustrates an exploded view of the syringe assembly of FIG. 1A.

FIG. 2A illustrates a perspective view of an embodiment of a plunger tip covering of the syringe assembly of FIG. 1A.

FIG. 2B illustrates a side cross-sectional view of the plunger tip covering of FIG. 2A.

FIG. 3A illustrates a perspective view of the plunger tip covering of FIG. 2A prior to assembly to a plunger of the syringe assembly of FIG. 1A.

FIG. 3B illustrates a perspective view of the plunger tip covering of FIG. 2A assembled to the plunger of the syringe assembly of FIG. 1A.

FIG. 4A illustrates a perspective view of another embodiment of a plunger tip covering of the syringe assembly of FIG. 1A.

FIG. 4B illustrates a side cross-sectional view of the plunger tip covering of FIG. 4A.

FIG. 5A illustrates a perspective view of the plunger tip covering of FIG. 4A prior to assembly to another embodiment of a plunger.

FIG. 5B illustrates a perspective view of the plunger tip covering of FIG. 4A assembled to the plunger of FIG. 5A.

FIG. 6 illustrates another embodiment of a plunger tip covering.

FIG. 7A is a cross-sectional view of an embodiment of a method of manufacture of a plunger wherein a plunger tip covering is formed.

FIG. 7B is a cross-sectional view of the method of manufacture of a plunger wherein a plunger tip is formed within a cavity of the plunger tip covering of FIG. 7A.

FIG. 7C is a cross-sectional view of the method of manufacture of a plunger wherein the plunger tip and plunger tip covering of FIG. 7B are coupled to a plunger handle.

### DETAILED DESCRIPTION

Coverings for syringe plunger tips, as well as syringes and syringe components having such coverings, are disclosed herein. In some embodiments, a covering is disposed on at least a portion of an external surface of a syringe plunger tip. The covering may comprise a material that is different than the material of the plunger tip. The material properties of the covering and the plunger tip may differ in at least one material property, as described more fully below. For example, the covering may comprise a material with a lower coefficient of friction when in contact with the interior surface of a syringe barrel than the material of the plunger tip. Further, in some embodiments, the plunger tip may compress to provide a sealing force between the plunger tip and covering and the interior surface of a syringe barrel.

The covering may further comprise a material that isolates, for example by physical and/or chemical separation, a plunger tip from a substance loaded into a reservoir defined by an interior surface of a syringe barrel. Again, this isolation may be due to prevention of physical contact between the plunger tip and the substance. Further, the covering may be substantially impermeable to migration of chemical components of the plunger tip across the covering.

In some embodiments, the covering comprises one or more polymeric materials. Syringe tip coverings within the scope of this disclosure may comprise polymeric materials including, but not limited to, linear low-density polyethylene (LLDPE), high-density polyethylene (HDPE), expanded polytetrafluoroethylene (ePTFE), polytetrafluoroethylene (PTFE), or a composite of any of these materials. Such coverings may facilitate, for example, controlled sliding of a syringe plunger tip along the length of an interior surface of a syringe barrel. Additionally or alternatively, such coverings may prevent a substance loaded within a syringe barrel from being contaminated by syringe components.

In some embodiments, a covering may prevent contact between a silicone plunger tip or a silicone lubricant and, for example, a substance containing polyvinyl alcohol (PVA) within the syringe barrel. In other embodiments, a covering may prevent contact between a silicone plunger tip or a silicone lubricant and a substance containing gelatin foam (examples include SurgiFoam™ from Ethicon or Gelfoam™ from Pfizer) within the syringe barrel. In still other embodiments, a covering may prevent contact between a silicone plunger tip or a silicone lubricant and a substance containing sodium tetradecyl sulfate. In some instances, silicone plunger tips or silicone lubricants may be incompatible with use of PVA or gelatin foam. In some instances, for example, silicone material from the plunger tip or silicone lubrication within the syringe barrel may tend to coat or otherwise adhere to the PVA, gelatin foam, or sodium tetradecyl sulfate foam if these components are in contact. This may, in turn, interfere with hydration of PVA or gelatin foam particles thus causing them to agglomerate or causing foam degradation. Thus, the coverings provided herein may be impermeable or demonstrate low permeability to silicone to prevent such contamination. As used herein, silicone refers broadly to polymeric compounds that consist of silicon-oxygen backbone chains ( . . . —Si—O—Si—O— . . . )ₙ (e.g., compounds containing one or more siloxane groups).

In some embodiments, the disclosed coverings include a covering locking member configured to couple with a plunger locking member to lock the covering over the plunger tip. When the covering is locked over the plunger tip, the covering is prevented from longitudinal and/or rotational movement relative to the plunger tip as the plunger is moved within the syringe barrel. In certain embodiments, the covering locking member includes a tab extending from the covering. The tab includes a hinge and a passage. The plunger includes a plunger locking member disposed adjacent or near a distal end. The plunger locking member includes a hook shaped member configured to be received by the passage of the tab when the covering is locked with the plunger to cover the plunger tip. In another embodiment, the covering locking member includes a tab having a foot portion and the plunger locking member includes a disk disposed adjacent or near a distal end. The tab is deflected radially inward when the foot portion engages with an interior surface of the syringe barrel as the plunger is inserted into the syringe barrel to lock the covering to the plunger and over the plunger tip.

Methods for manufacturing a syringe assembly including a covering for a syringe plunger tip are also provided by the present disclosure. In some embodiments, the methods comprise coupling or locking a covering to a plunger to cover a syringe plunger tip. The covering may be locked or coupled to the plunger via the covering locking member and the plunger locking member. In some embodiments, the covering may be formed using an injection molding coining technique.

It will be readily understood with the aid of the present disclosure that the components of the embodiments, as generally described and illustrated in the figures herein, could be arranged and designed in a variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

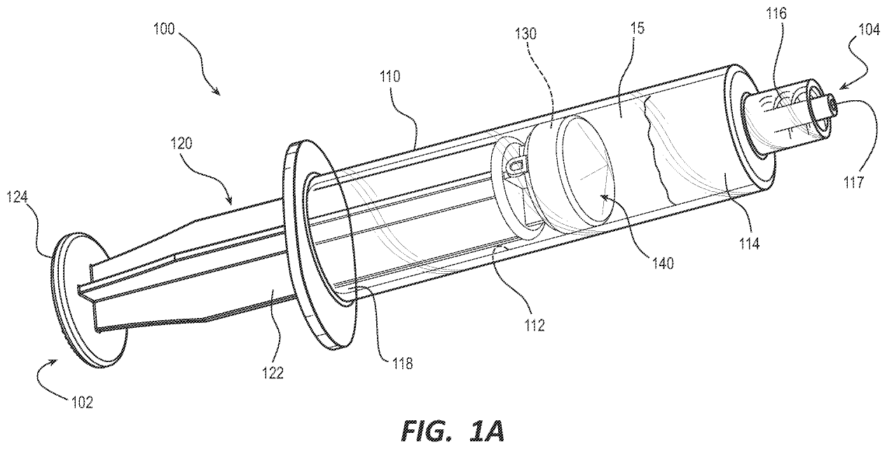

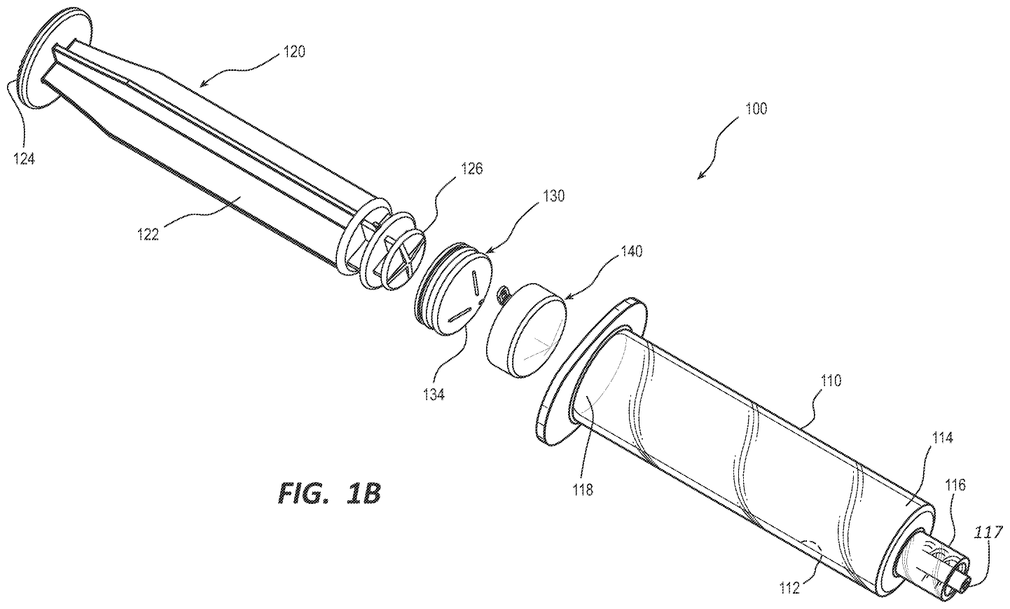

FIGS. 1A and 1B are, respectively, a perspective view and an exploded view of one embodiment of a syringe assembly 100. In the illustrated embodiment, the syringe assembly 100 extends between a proximal end 102 and a distal end 104. Further, the illustrated syringe assembly 100 includes a syringe barrel 110 having an interior surface 112. The interior surface 112 may define a reservoir 114. The reservoir 114 defined by interior surface 112 may be any suitable size and shape, e.g., for receiving a syringe plunger (such as a plunger 120 discussed below). In the illustrated embodiment, the reservoir 114 may be generally cylindrical in shape. The reservoir 114 defined by the interior surface 112 may be configured to receive a substance 15. The syringe barrel 110 may further include a tip 116 having an opening 117 in communication with the reservoir for receiving the substance 15 into or expelling the substance 15 from the reservoir 114. In the illustrated embodiment, the tip 116 and opening 117 are disposed adjacent a distal end of the syringe barrel 110. The syringe barrel 110 may include an additional opening 118 located, for example, at the proximal end of the syringe barrel 110, for receiving the plunger 120, which may be disposed or partially disposed within the syringe barrel 110. The plunger 120 may comprise an elongate handle portion 122 having both a proximal end 124 and a distal end 126. The plunger 120 may typically have a length that is at least as long as a length of the syringe barrel 110. Accordingly, the proximal end 124 of the plunger may extend proximally out from the syringe barrel 110 when the plunger 120 is disposed within the syringe barrel 110. The plunger 120 may otherwise have any size and shape that is suitable for being disposed within the syringe barrel 110.

A plunger tip 130 may be coupled to the handle portion 122. The plunger tip 130 may be a tip that is integrally molded with handle portion 122 or a tip that is attached separately. In one embodiment, the plunger tip 130 may be selectively coupled to the distal end 126 of the handle portion 122. The plunger tip 130 may comprise an external surface 134. In certain embodiments, the plunger tip 130 may be configured to contact at least a portion of the interior surface 112 of barrel 110. Accordingly, the plunger tip 130 may have a size and shape that is complimentary to the size and shape of the reservoir 114 defined by interior surface 112. For example, the plunger tip 130 may be generally cylindrical in shape such that a portion of its external surface 134 may have a diameter that is the same or larger in size to a diameter of the interior surface 112 of a syringe barrel 110 that is also cylindrical in shape.

Furthermore, the plunger tip 130 may be sized such that, when disposed within the syringe barrel 110, the syringe barrel 110 radially constrains and/or compresses the plunger tip 130. The plunger tip 130 may be comprised of an elastomeric material that, due to the compression, provides a radially outward-oriented force on the interior surface 112 of the syringe barrel 110. Compression of the plunger tip 130 may thus result in a radial outward force that tends to seal the plunger tip 130 (including a plunger tip covering 140 as discussed below) against the interior surface 112 of the syringe barrel 110.

As illustrated the plunger tip covering 140 may be disposed on the external surface 134 of the plunger tip 130. In certain embodiments, the covering 140 may be disposed on the entire external surface 134 of plunger tip 130. In other embodiments, the covering 140 may be disposed on only a limited portion of the external surface 134 of the plunger tip 130. In certain embodiments, the covering 140 may be disposed on the external surface 134 such that it is positioned between at least a portion of the external surface 134 of the plunger tip 130 and the interior surface 112 of barrel 110. In such instances, the covering 140, rather than the external surface 134, will be in direct contact with the interior surface 112. The covering 140 may further be disposed on the external surface 134 of plunger tip 130 such that it is positioned between the external surface 134 and reservoir 114. In that way, the covering 140 may provide a barrier between the external surface 134 and a substance 15 loaded in the reservoir 114 such that there is no physical contact between the external surface 134 and substance 15.

The covering 140 may be compliant or flexible such that the covering 140 compresses and expands with the plunger tip 130. In some embodiments, the covering 140 may flex with the plunger tip 130 such that when the plunger tip 130 is compressed within the syringe barrel 110, the plunger tip 130 exerts a force radially outward on the covering 140, causing the covering 140 to seal against the interior surface 112 of the syringe barrel 110. In some instances, the plunger tip 130 may provide resiliency and/or compressibility to the plunger tip 130-covering 140 assembly while the covering 140 provides a friction bearing surface against the interior surface 112 of the barrel 110 and a contact surface for the substance 15 within the barrel 110.

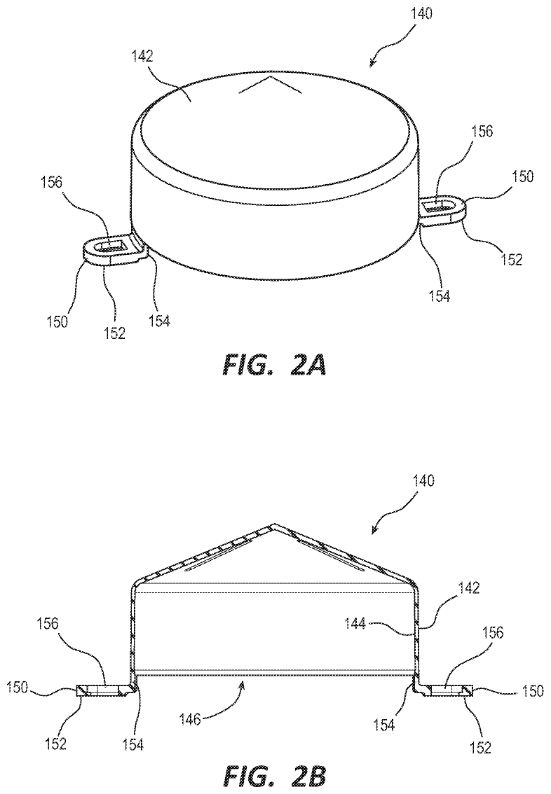

Referring to FIGS. 2A and 2B, a perspective view and a cross-sectional side view of the covering 140 are shown. The covering 140 may include an outer surface 142 and an inner surface 144. The inner surface 144 may define a cavity 146 for receiving the plunger tip 130. In some embodiments, ridges or detents may protrude from the inner surface 144 for engaging features of the plunger tip 130 and for holding the covering 140 in position on the plunger tip 130 via, for example, a friction fit.

The covering 140 includes a covering locking member 150 coupled to and extending from a proximal edge of the covering 140. The covering locking member 150 may be used to lock the covering 140 onto the plunger 120 over the plunger tip 130. The depicted covering locking member 150 includes a tab 152 and a hinge 154. The hinge 154 flexibly couples the tab 152 to the covering 140. The hinge 154 can be a living hinge or any other suitable type of hinge capable of flexing at least 90 degrees. The tab 152 includes a passage 156 extending through a portion of the tab 152. The passage 156 is shaped and sized to receive a plunger locking member 170, as will be described below. In other embodiments, the tab 152 may include any suitable feature capable of engaging with a mating feature of the plunger locking member 170. In certain embodiments, the covering locking member 150 may include a tether disposed between the tab 152 and the covering 140 and used to flexibly couple the tab 152 to the covering 140. The tether can be of any suitable length.

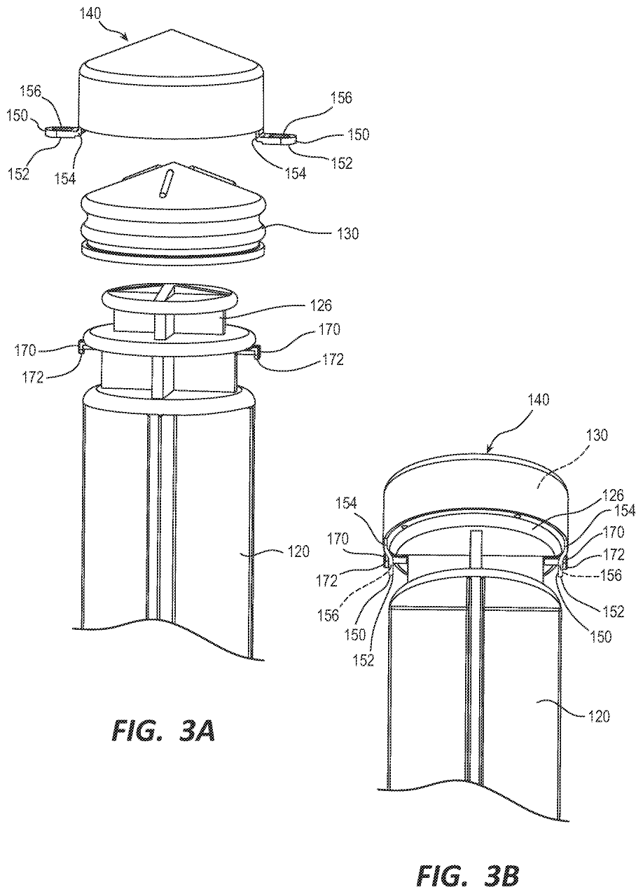

FIG. 3A illustrates a distal portion of the plunger 120, the plunger tip 130, and the covering 140 in a pre-assembled state. As illustrated, the tab 152 of the covering locking member 150 extends radially outward from the covering 140. The plunger 120 includes a plunger locking member 170 disposed adjacent the distal end 126 of the plunger 120. As depicted, the plunger locking member 170 includes a hook 172 configured to be received by the passage 156 of the tab 152. In other embodiments, the plunger locking member 170 may include any other suitable feature capable of engaging a mating feature of the covering locking member 150. For example, the plunger locking member 170 and the covering locking member 150 may include a ball-and-socket mating feature or an undercut annular ring, for example. Additionally, other mating features are contemplated within the scope of the disclosure.

FIG. 3B illustrates the distal portion of the plunger 120, the plunger tip 130, and the covering 140 in an assembled locked state. As illustrated, the plunger tip 130 is coupled to the plunger 120 and the covering 140 is disposed over the plunger tip 130 such that the plunger tip 130 is substantially covered by the covering 140. The tab 152 of the covering locking member 150 is flexed proximally about the hinge 154. The hook 172 of the plunger locking member 170 is disposed in the passage 156 to lock the covering 140 over the plunger tip 130 and onto the plunger 120 to prevent the covering 140 from longitudinal and rotational movement relative to the plunger tip 130. In other words, coupling of the covering locking member 150 with the plunger locking member 170 facilitates longitudinal and rotational movement of the covering 140 with longitudinal and rotational movement of the plunger 120 within the syringe barrel 110.

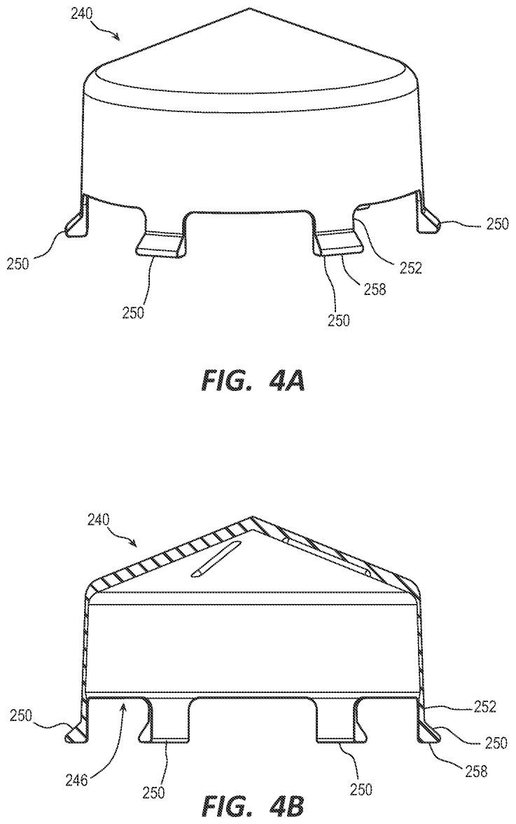

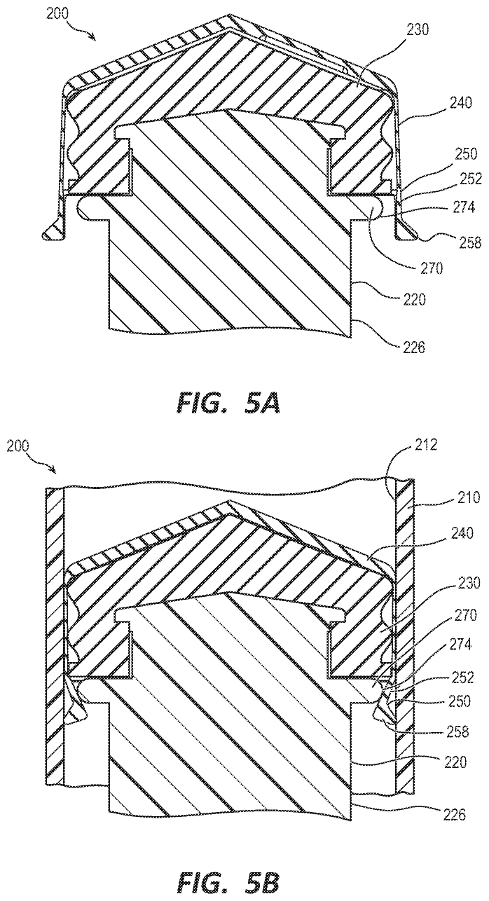

FIGS. 4A-5B are a series of views of another embodiment of a syringe assembly and components thereof that can, in certain respects, resemble a syringe assembly and components thereof described in connection with FIGS. 1A-3B. It will be appreciated that all the illustrated embodiments may have analogous features. Accordingly, like features are designated with like reference numerals, with the leading digits incremented to “2.” For instance, the covering is designated as “140” in FIGS. 1A-3B, and an analogous covering is designated as “240” in FIGS. 4A-5B. Relevant disclosure set forth above regarding similarly identified features thus may not be repeated hereafter. Moreover, specific features of the syringe assembly and related components (e.g., the covering) shown in FIGS. 1A-3B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the syringe assembly and components thereof of FIGS. 4A-5B. Any suitable combination of the features, and variations of the same, described with respect to the syringe assembly and components thereof illustrated in FIGS. 1A-3B can be employed with the syringe assembly and components thereof of FIGS. 4A-5B, and vice versa. This pattern of disclosure applies equally to further embodiments depicted in subsequent figures and described hereafter.

Referring to FIGS. 4A and 4B, a perspective view and a cross-sectional side view of a plunger tip covering 240 are shown. The covering 240 includes a covering locking member 250 coupled to and extending from a proximal edge of the covering 240. The covering locking member 250 may be used to lock the covering 240 onto a plunger over a plunger tip. The depicted covering locking member 250 includes a plurality of tabs 252 spaced equidistant around a perimeter of the covering 240. The number of tabs 252 may be two, three, four, five, six, or more. In the depicted embodiment, the number of tabs 252 is eight. Each of the tabs 252 includes a foot shaped portion 258 configured to engage a plunger locking member 270, as described below.

FIG. 5A illustrates a cross-sectional view of a distal portion of a plunger 220, a plunger tip 230, and the covering 240 of a syringe assembly 200 in an assembled state. As illustrated, the tab 252 of the covering locking member 250 extends from the covering 240. The plunger 220 includes a plunger locking member 270 disposed adjacent the distal end 226 of the plunger 220. As depicted, the plunger locking member 270 includes a disk 274 configured to be engage the tab 252 when the tab 252 is radially inwardly deflected.

FIG. 5B illustrates the distal portion of the plunger 220, the plunger tip 230, and the covering 240 of the syringe assembly 200 in a locked state. As illustrated, the plunger tip 230 is coupled to the plunger 220 and the covering 240 is disposed over the plunger tip 230 such that the plunger tip 230 is substantially covered by the covering 240. The covering 240 is disposed within a syringe barrel 210. The tab 252 of the covering locking member 250 is flexed radially inward by an interior surface 212 when the foot shaped portion 258 engages with the interior surface 212. An interior surface of the tab 252 engages with a distal surface of the disk 274 to lock the covering 240 onto the plunger tip 230 and prevent disengagement of the covering 240 from the plunger 220 as the plunger 220 is moved within the syringe barrel 210.

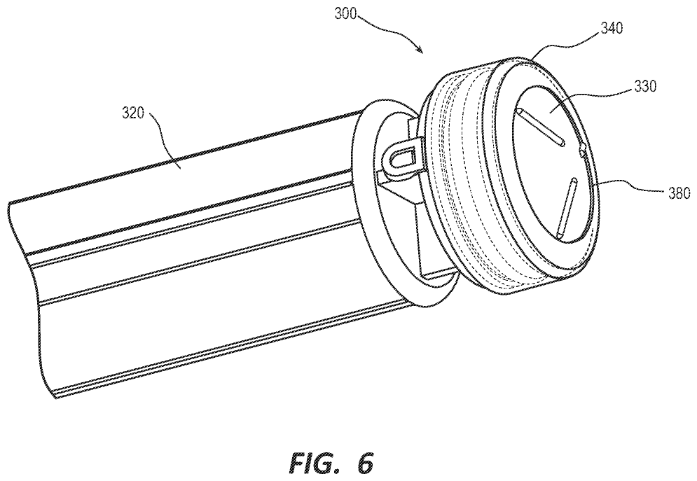

FIG. 6 illustrates another embodiment of a portion of a syringe assembly 300. As illustrated, the syringe assembly 300 comprises a plunger 320, a plunger tip 330, and a covering 340. The covering 340 includes an opening 380 at a proximal end of the covering 340. The opening 380 can expose at least a portion of a distal surface of the plunger tip 330. In some such embodiments, the covering 340 may comprise a shrink sleeve, meaning a sleeve or cylinder of material configured to be shrunk (e.g. heat shrink material) around the plunger tip 330 to couple the covering 340 to the plunger tip 330. In such embodiments, the sealing or compression rings along the outside diameter of the plunger tip 330 may be covered by the covering 340, and thus disposed at the interface between the plunger tip 330 and a syringe barrel without covering the entire plunger tip 330.

The syringe barrel of any of the embodiments of the present disclosure may comprise any size, shape and material known in the art to be suitable for such syringe components. Accordingly, the syringe barrel may have a cylindrical shape and may be configured to have a plunger disposed within an interior reservoir of the syringe barrel. In some embodiments, the syringe barrel may comprise a plastic or polymeric material.

The handle portion of the plunger of any of the embodiments of the present disclosure may comprise any size, shape, and material known in the art to be suitable for such syringe components. The handle portion may comprise a stiff or rigid material that can transfer a force exerted upon it to a plunger tip to which it is coupled or attached to move the plunger tip within the syringe barrel. In some embodiments, the handle portion may comprise a plastic or polymeric material. The handle portion may be longer in its length than in its width or diameter such that it can extend through the entire length of a syringe barrel. Such an elongate design allows the handle portion to be used to move a plunger tip throughout the entire length of a syringe barrel.

The plunger tip may be a tip that is integrally molded to the plunger handle portion or a tip that is attached separately. The plunger tip may be of any design that is of a suitable size and shape for being disposed or otherwise inserted into a syringe barrel such that the plunger tip is in mechanical communication with an interior surface of the syringe barrel. For example, the plunger tip may have a generally cylindrical or circular shape, and the syringe barrel may have a generally cylindrical or circular shape such that they are in contact with each other along all or a portion of an outer circumference of the plunger tip and an inner circumference of the syringe barrel. Further, the plunger tip may comprise an entirely solid structure or may comprise a structure that is hollow or includes a cavity.

In some embodiments, the plunger tip may comprise a material that is resiliently compressive such that the plunger tip may exert a force against the interior surface of the syringe barrel. Thus, the plunger tip may be capable of compressing and deforming against the interior surface of the syringe barrel when disposed within the interior of the syringe barrel. The force exerted by the resiliently compressive material deforming against the interior surface may serve to hold the plunger tip in position within the syringe barrel until displaced by a practitioner. The force may also be a sealing force that prevents any leakage or transmission past the plunger tip of a substance loaded within a reservoir defined by an interior surface of the syringe barrel. In some embodiments, the plunger tip may comprise a material containing silicone, such as, for example, silicone oil or silicone rubber.

Any of the components of the syringes disclosed herein (e.g., the barrel, plunger, handle portion and/or plunger tip) may comprise glass or any polymeric material, such as, for example, acrylonitrile-butadiene-styrene (ABS) polymer, polycarbonate (PC), polypropylene, or cyclo-olefin polymer or copolymer, and/or combinations or blends of these materials such as PC-ABS. In some embodiments, any of the syringe components disclosed herein, including the plunger tip, may comprise a material that contains silicone, such as, for example, silicone oil or silicone rubber. Alternatively, any of the syringe components disclosed herein may comprise a silicone-free material. Additionally, any of the syringe components disclosed herein may be lubricated with a silicone-free lubricant.

The covering according to the present disclosure may comprise a different material than that of the plunger tip. In some embodiments, the material properties of the materials used for the covering and the plunger tip may differ in certain respects. Accordingly, the covering may comprise any suitable material including those that provide a reduced-friction interface, as compared to the plunger tip material, between a plunger tip and an interior surface of a syringe barrel. Thus, the covering may comprise a material that provides a lower coefficient of friction between the covering and the interior surface of the syringe barrel than the coefficient of friction between the plunger tip and the interior surface of the syringe barrel.

Accordingly, in some embodiments the use of a covering as disclosed herein may reduce or eliminate the need to use a lubricant (e.g., a lubricant containing silicone) within the syringe assembly (e.g., within the syringe barrel), which, in turn, may prevent such a lubricant from contaminating or otherwise affecting a substance held within the syringe. For example, in some instances silicone lubricants may coat or otherwise interfere with the hydration of PVA or gelatin foams and foaming of sodium tetradecyl sulfate. Coverings used in place of silicone lubricant may therefore be configured for use with PVA, gelatin foams, or sodium tetradecyl sulfate foams. Additionally or alternatively, in certain embodiments, a covering as disclosed herein may be used to achieve a specific breakaway force with respect to the movement of a plunger tip through a syringe barrel. This break away force may be between 0.5 pound of force (lbf) and 3 lbf, 1 lbf and 2 lbf, less than 3 lbf, less than 2 lbf, less than 1.5 lbf, or less than 1 lbf. Additionally, in some embodiments, a covering as disclosed herein may be configured such that coefficients of static and dynamic friction between the covering and a surface of the syringe assembly (e.g., the interior surface of the syringe barrel) are closely matched, for example, within 5% or within 2% of each other, which may facilitate smooth or even use of the syringe.

The covering of the present disclosure may also comprise a material that isolates a plunger tip from a substance loaded into a reservoir defined by an interior surface of a syringe barrel by preventing physical contact between the plunger tip and the substance. Thus, the covering may comprise a material that is impermeable to either the substance or to a material contained in the plunger tip. For example, the covering may prevent a silicone-containing material, such as silicone rubber, of a plunger tip from contaminating a substance comprising PVA, gelatin foam, or sodium tetradecyl sulfate loaded in the syringe.

In some embodiments, the covering may comprise one or more polymeric materials. For example, the covering may comprise LLDPE, HDPE, PTFE, epTFE, or a composite of any of these materials. In certain embodiments, the plunger tip may comprise silicone and the covering disposed on the plunger tip may comprise LLDPE, HDPE, PTFE, ePTFE, or a composite of any of these materials.

Methods for manufacturing a syringe assembly are also provided by the present disclosure. Referring again to FIGS. 1A-3B, one embodiment of such a method may comprise forming the covering 140 using an injection molding technique where a molten material is injected into a cavity of a mold having a shape of the covering 140. In some embodiments, the injection molding technique may comprise coining or injection-compression molding where a compressive force applied by movement of the mold reduces the cavity thickness to reduce a wall thickness of the covering 140. The covering 140 can be disposed over the plunger tip 130 and coupled to the plunger 120. Such coupling may comprise coupling the covering locking member 150 to the plunger locking member 170 where the passage 156 of the tab 152 is disposed over the hook 172. In another embodiment illustrated in FIGS. 4A-5B, such coupling may comprise inserting the covering 240 and the plunger 220 into the syringe barrel 210 such that the tab 252 is deflected radially inward and the foot shaped portion 258 engages with the disk 274.

The methods disclosed may further comprise coupling the plunger tip 130 to the plunger handle 122. In certain embodiments, the plunger tip 130 may be coupled to the distal end 126 of the handle portion 122. The methods may also comprise loading the substance 15 into the reservoir 114. The substance 15 may be loaded, e.g., through the opening 117 of the tip 116 of the syringe barrel 110 that is in communication with the reservoir 114. In some embodiments, the substance 15 may comprise PVA, gelatin foam, or sodium tetradecyl sulfate.

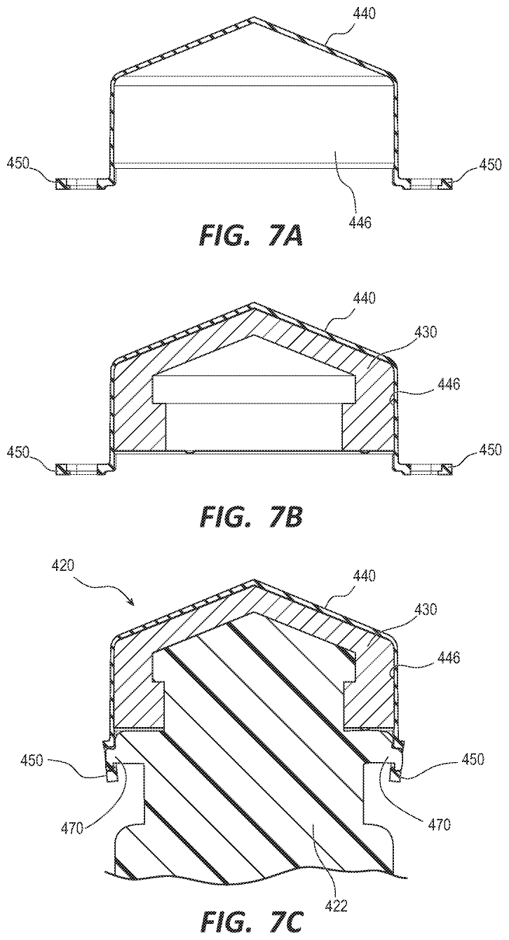

FIGS. 7A-7C illustrate another method of manufacture of a syringe assembly. In the illustrated embodiment, the method may comprise forming a plunger tip covering 440 using an injection molding technique where a molten material is injected into a cavity of a mold having a shape of the plunger tip covering 440, as shown in FIG. 7A. In another embodiment, a plunger tip covering substantially similar to the plunger tip covering 240 previously described or any other suitable plunger tip covering may be formed using an injection molding technique. A plunger tip 430 can be formed within the plunger tip covering 440 as shown in FIG. 7B. The plunger tip covering 440 can be formed by an injection molding or over molding or two-shot injection molding process of a thermoplastic elastomer (TPE) material wherein the TPE material is injected directly into a cavity 446 of the plunger tip covering 440. In certain embodiments, the TPE material may have a hardness ranging from about 30 Shore A durometer to about 70 Shore A durometer. Removal of the plunger tip 430 from the plunger tip covering 440 may be prevented by adhesion of the material of the plunger tip 430 to the material of the plunger tip covering 440 and/or by vacuum or negative pressure formed between an outer surface of the plunger tip 430 and an inner surface of the plunger tip covering 440 when the plunger tip 430 is displaced relative to the plunger tip covering 440. The plunger tip 430 can be disposed over a distal end 426 of the plunger handle 422 and the plunger tip covering 440 coupled to the plunger handle 422, as shown in FIG. 7C, to form a plunger 420. Such coupling may comprise coupling a covering locking member 450 to a plunger locking member 470 as previously described.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of manufacture of a syringe assembly may include one or more of the following steps: coupling a plunger tip to a plunger handle; disposing a plunger tip covering over the plunger tip; and coupling a covering locking member to a plunger locking member to lock the plunger tip covering onto the plunger handle and over the plunger tip. Other steps are also contemplated. Another example of a method of manufacture of a syringe assembly may include one or more of the following steps: forming a plunger tip covering; forming a plunger tip within a cavity of the plunger tip covering; coupling the plunger tip to a plunger handle; and coupling a covering locking member to a plunger locking member to lock the plunger tip covering onto the plunger handle and over the plunger tip.

While specific embodiments of coverings for syringe plunger tips and methods related thereto have been described, it is to be understood that the disclosure provided is not limited to the precise configuration and components disclosed. Various modifications, changes, and variations apparent to those of skill in the art having the benefit of this disclosure may be made in the arrangement, operation, and details of the devices, methods, and systems disclosed.

Furthermore, any reference to “one embodiment,” “an embodiment,” or “the embodiment,” as used throughout this disclosure, means that a particular feature, structure, or characteristic described in connection with that embodiment is included in at least one embodiment. Thus, the quoted phrases, or variations thereof, as recited throughout this specification are not necessarily all referring to the same embodiment.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use. As specifically applied to a syringe assembly of this disclosure, the proximal end of the syringe assembly refers to the end nearest to the practitioner and the distal end refers to the opposite end, the end nearest to the patient.

The term “material property” refers to any property or characteristic of a substance comprising components described herein. For example, material properties may include, but are not limited to, a material's chemical make-up (e.g., materials that contain silicone versus those that do not), a material's coefficient of friction as measured with another material or surface, a material's permeability, and other physical, chemical, mechanical, structural, electrical, or other properties.

References to approximations are made throughout this specification, such as by use of the term “substantially.” For each such reference, it is to be understood that, in some embodiments, the value, feature, or characteristic may be specified without approximation. For example, where a qualifier such as “substantially” is used, this term includes within its scope the qualified words in the absence of their qualifiers. For example, where the term “substantially covered” is recited with respect to a feature, it is understood that in further embodiments, the feature can have a fully covered configuration.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A syringe comprising:
a barrel comprising an interior surface defining a reservoir; and
a plunger assembly comprising:
a plunger handle comprising a proximal end and a distal end;
a plunger tip coupled to the distal end;
a plunger locking member disposed adjacent the distal end; and
a plunger tip covering at least partially disposed over the plunger tip, the plunger tip covering comprising a covering locking member couplable to the plunger locking member to lock the plunger tip covering to the plunger handle and over the plunger tip.

2. The syringe of claim 1, wherein the covering locking member comprises a tab extending from a proximal end of the plunger tip covering.

3. The syringe of claim 2, wherein the tab comprises a tether coupled to the plunger tip covering.

4. The syringe of claim 2, wherein the tab comprises:
a hinge coupled to the plunger tip covering; and
a passage extending through the tab.

5. The syringe of claim 4, wherein the plunger locking member comprises a hook shaped member.

6. The syringe of claim 5, wherein the passage is sized to receive the hook shaped member.

7. The syringe of claim 2, wherein the tab comprises a foot portion.

8. The syringe of claim 2, wherein the plunger locking member comprises a disk disposed adjacent the distal end of the plunger handle; and
wherein the plunger locking member is coupled to the foot portion of the tab when the plunger assembly is inserted into the barrel.

9. The syringe of claim 1, wherein the plunger tip covering comprises a distal opening to expose a distal portion of the plunger tip.

10. The syringe of claim 1,
wherein the plunger tip comprises a first material,
wherein the plunger tip covering comprises a second material, and
wherein a coefficient of friction between the second material and the interior surface is lower than a coefficient of friction between the first material and the interior surface.

11. The syringe of claim 10, wherein the second material comprises linear low-density polyethylene (LLDPE).

12. A syringe plunger assembly comprising:
a plunger comprising:
an elongate handle portion defining a proximal end and a distal end;
a plunger tip coupled to the distal end of the elongate handle portion; and
a plunger locking member; and
a plunger tip covering at least partially disposed on an external surface of the plunger tip, the covering comprising a covering locking member couplable to the plunger locking member to lock the plunger tip covering onto the plunger tip.

13. The syringe plunger assembly of claim 12, wherein the covering locking member comprises a tab extending from a proximal end of the plunger tip covering.

14. The syringe plunger assembly of claim 13, wherein the tab comprises a tether coupled to the plunger tip covering.

15. The syringe plunger assembly of claim 13, wherein the tab comprises:
a hinge coupled to the plunger tip covering; and
a passage extending through the tab.

16. The syringe plunger assembly of claim 13, wherein the tab comprises a foot portion.

17. The syringe plunger assembly of claim 16,
wherein the plunger locking member comprises a locking disk disposed adjacent the distal end of the elongate handle; and
wherein the locking disk is coupled to the foot portion of the tab when the plunger is inserted into a syringe barrel.

18. A method of manufacture of a syringe assembly comprising:
coupling a plunger tip to a plunger handle;
disposing a plunger tip covering over the plunger tip; and
coupling a covering locking member to a plunger locking member to lock the plunger tip covering onto the plunger handle and over the plunger tip.

19. The method of claim 18, wherein coupling the covering locking member to the plunger locking member comprises:
proximally bending a tab of the covering locking member; and
disposing a hook of the plunger locking member into a passage of the tab.

20. The method of claim 18, wherein coupling the covering locking member to the plunger locking member comprises:
radially inwardly bending a foot portion of the covering locking member when the plunger tip covering is inserted into a syringe barrel; and
coupling the foot portion with a locking disk of the plunger locking member.
